(19) United States
(12) Patent Application Publication
(10) Pub. No.: US 2013/0204397 A1
(43) Pub. Date: Aug. 8, 2013

(54) PROSTHETIC FOOT COVERING ENABLING RAPID CONVERSION BETWEEN SHOE AND BAREFOOT WALKING

(71) Applicant: Elwin Isaac Nordman, JR., San Jose, CA (US)

(72) Inventor: Elwin Isaac Nordman, JR., San Jose, CA (US)

(21) Appl. No.: 13/365,046

(22) Filed: Feb. 2, 2012

(51) Int. Cl. A61F 2/66

(52) U.S. Cl. 623/53

(57) ABSTRACT

A covering for a prosthetic leg. The prosthetic leg is nominally adjusted for optimal performance while the user is wearing shoes. The covering is configured to cosmetically resemble skin, and to shield some or all of the prosthetic leg mechanical components from outside view, thus making the prosthetic leg resemble a natural leg. The covering is designed to enable a prosthetic leg wearer to walk without shoes in a manner that makes it look as if the wearer is walking barefoot. To avoid prosthetic leg misadjustment problems such as hyperextended knee or prosthetic posterior lean, the covering is further configured with one or more internal inserts designed to adjust the height of the prosthetic foot to compensate for the missing elevation caused by the missing shoe. The device may be further configured with one or more fasteners to facilitate rapid application and removal.

### BACKGROUND OF THE INVENTION

1. Field of the Invention

This invention is in the field of prosthetics and prosthetic coverings for lower limb amputee patients.

2. Description of the Related Art

Human walking is a complex cyclical series of body movements in which the weight of the body is sequentially supported by first one leg and then the other. As the weight of the body is supported by one leg during the stance phase portion of the walking or gait cycle, the other leg swings forward so that it in turn can be later used for weight support during a later part of cycle. During this gait cycle, the pelvis both rotates and lists. At the knee joint, the knee extension angle changes so that at the beginning and end of the stance phase, the knee is nearly fully extended (e.g. the angle of the knee is nearly 180°), while at the middle of the stance phase, the knee is more flexed.

The angle of the ankle and foot also change during the gait cycle. At the beginning of the stance phase, the foot flexes about the ankle so that the heel of the foot strikes the floor first. During the middle of stance phase, the angle of the foot then flattens, relative to the floor. During the later part of the stance phase, the foot then rotates further relative to the ankle, so that the toe of the foot remains in contact with the floor while the heel elevates. These various bone and joint movements are controlled by various muscles that typically operate both above and below their respective joints. These muscles in turn are under precise control by nerves and the body's various natural sensors.

Amputation literally cuts through this delicate biomechanical mechanism with a knife, and throws the entire process out of kilter.

There are various types of lower limb amputations. In some cases, only one limb is amputated (unilateral amputation), while in other cases both limbs are amputated (bilateral amputation). In this discussion, lower limb amputation will be assumed to mean that at least the natural foot and ankle has been removed. Whenever possible, surgeons will attempt to preserve the patient's natural knee joint, and lower limb amputees who retain their natural knee joint and at least some of the natural tibia bone are called below-knee amputees or “transtibial” amputees. By contrast, when the knee and portions of the femur must be amputated, these amputees are termed “transfemoral” or above knee (mid thigh) amputees. In either case, either during the initial amputation surgery, or in later subsequent surgeries, the remaining bone, muscle and skin at the severed end of the limb are configured into a stump that in turn can be fit into the weight being socket of an appropriate lower-limb prosthetic device.

As might be imagined, lower limb amputation greatly interferes with the complex series of events that occur during human walking, and much of the art and science of lower limb prosthetic design has been focused on providing artificial mechanisms that, working in concert with the amputee's remaining natural muscles and tissues, can provide as adequate a substitution for a natural gait as possible.

Although the technology of lower limb prosthetics design has advanced to the point where, in many cases, the performance of the prosthetic limb can be remarkably natural, the limitations of even modern prosthetics still must be appreciated. The lower limb amputee has to walk without the aid of many important muscle groups, and without the aid of his or her natural lower extremity nerves and natural kinesthetic sensors (e.g. sense of touch, kinesthetic senses, and the like). The amputee's lower limb stump or stumps have to bear weight in a biologically unnatural manner, and can be damaged if not treated carefully. As a result, prosthetics specialists must precisely adjust the amputee's lower limb prosthetics to various parameters, including the height characteristics of the shoes that the prosthetic patient intends to wear.

Misadjusted or misconfigured lower limb prosthetics can produce a number of problems. One problem, for example, is the problem of knee joint hyperextension. In a knee joint hyperextension situation, particularly during the initial part of the stance phase, the angle of the knee is overextended beyond 180°, thus imposing a very unnatural stretching force on the knee.

This problem is particularly acute for transtibial amputees who have retained their natural knee joint or joints, because knee hyperextension can damage precious natural tissue. However even for trans-femoral amputees, knee hyperextension moments are undesirable as well as they create difficulty in walking and pain in the hip region.

Such knee joint hyperextension often occurs when, for example, an amputee attempts to walk barefoot on a lower limb prosthetic that has been adjusted for walking with shoes with a certain shoe heel height.

Another problem that can occur with lower limb amputees when walking shoeless with a lower limb prosthetic otherwise configured for walking with shoes is the problem of prosthetic posterior lean. Here the lower limb prosthetic, normally adjusted for walking with shoes with a certain shoe heel height, tends to fall or lean backwards while standing barefoot. This again can cause damage to the amputee's remaining natural tissue, as well as increasing the risk of a fall.

As a result of these and other considerations, lower limb amputees are typically given prosthetics adjusted for wear with particular type of shoes with a particular shoe height and heel height. Lower limb amputees are further instructed by their prosthetics clinicians to always wear shoes with the same approximate shoe and shoe heel height, and to not attempt to either walk barefoot (i.e. without shoes), or to attempt to walk in shoes with a substantially different height and shoe heel height.

### Enhancing the Cosmetic Appearance of Lower Limb Prosthetics:

Changing for the moment to a different type of prosthetics problem, lower limb prosthetics are typically designed from a functional mechanical engineering basis, and often their unadorned cosmetic appearance looks mechanical and very unnatural. To help improve this cosmetic appearance, the artificial foot of a lower limb prosthetic will often be sold with the mechanical portions of the artificial foot covered by a foot shell, which is a relatively thick, skin-colored plastic covering, molded in the general shape of a natural foot (i.e. often with toes) designed to allow the artificial foot to fit into the shoe that the lower limb amputee intends to wear. This foot shell will often terminate at about the ankle level of the prosthetic foot, exposing various mechanical shafts, bolts, and other fixtures above the shell to the outside world.

In many lower limb prosthetics, the region of the prosthetic foot in between the ankle and the socket will be left in an unadorned open state that exposes the underlying mechanics of the prosthetic foot. This makes it easier to maintain and adjust the prosthetic foot, but of course makes it visually obvious that the lower limb amputee has a prosthetic foot.

In order to better shield the inner mechanism of a lower limb prosthetic, various types of prior art “skin” or “covering” devices have been proposed.

For example, Garcia, in U.S. Pat. No. 3,400,408, proposed an elastic cover for a particular prosthetic leg design comprised of a mass of polyurethane foam material and a latex cover.

Chen, in U.S. Pat. No. 5,133,775, proposed a covering device for an artificial limb made from a semi-rigid polyurethane foam with a screen-like flexible portion in the knee region which presumably made it easier for the covering to accommodate flexation in artificial knee joints for trans-femoral amputees.

Ahlert, in U.S. Pat. No. 5,593,453 proposed a prosthesis cover, apparently intended for use in the rain or other wet environments, with a waterproof sheet material, antiskid surface, finger loops to facilitate mounting, and internal ribs to better grip the underlying prosthetic limb.

Marquette, in U.S. Pat. No. 6,153,139 proposed a method of making a prosthesis covering apparatus intended to make a prosthetic more natural and lifelike. This method produced a protective and cosmetic covering intended to be stretched over a prosthetic foot, and resulted in a more aesthetically-pleasing covering for the prosthetic foot.

Laghi, in U.S. Pat. No. 6,911,049, proposed cosmetic covers for prosthetic limbs. These covers were formed from a heat-shrinkable plastic so that the cover could be applied to the prosthetic limb, and then shrunk fit to adhere better to the shape of the prosthetic foot.

Various such coverings or skins are presently commercially available from various companies. For example, RSLSteeper, a UK company, and their US affiliate SteeperUSA produce the SKINERGY PLUS™ line of silicone polymer based cosmesis covering for lower limb prosthetics. These coverings are available in 19 different color shades, are made from a reinforced mesh and silicone material, and can cover a prosthetic foot from the bottom to about a maximum top distance of 66 cm.

The company also provides a custom silicone service that produces silicone prosthetic coverings designed to mimic the translucent multi-colored nature of real human skin, and which is individually cast and color matched to the amputee's own natural skin, producing a very lifelike silicone covering that can then be placed over the prosthetic limb on a usually long-term basis.

### BRIEF SUMMARY OF THE INVENTION

Unfortunately, although from a functional standpoint, it is often inadvisable for a lower limb amputee to walk “barefoot”, that is to attempt to walk without shoes on a lower limb prosthetic adjusted for shoes, socially this is sometimes hard to avoid.

For example, in some households, such as many Asian households, it is considered rude to wear shoes indoors. A lower limb amputee confronted with such a situation is presently at a loss as to what to do. It is difficult to knowingly enter into a situation where the risk of damage and falls is significant. Even if the lower limb amputee were to assume the risk of complications, such as knee hyperextension and prosthetic posterior lean, without the shoe, the mechanical nature of the prosthetic limb becomes more clearly revealed, leading to more social awkwardness.

In other situations, such as poolside parties, athletic clubs, and the like, prosthetic users are under social pressure both to walk barefoot, and also to expose more skin than normal. Here again, these situations expose the lower limb amputee to both risk of damage due to knee hyperextension and prosthetic posterior lean, as well as and more social awkwardness due to more prominent exposure of the mechanical nature of the prosthetic.

The invention is based, in part, on the insight that what is needed is a new or improved type of lower limb prosthetic covering that can be rapidly put over a lower limb prosthetic previously adjusted for walking in a shoe in such situations, and would automatically compensate for height and heel differences caused by this missing shoe. Ideally this covering would cosmetically resemble natural skin as well, thus drawing attention away from the artificial nature of the prosthetic.

Thus the invention's prosthetic covering device would, on the one hand, be designed to compensate for the differences in heel height and other parameters caused by the missing shoe, while on the other hand also be designed to closely resemble the outside of a normal foot. This would enable a lower limb amputee to both avoid risk of damage while walking barefoot (e.g. without shoes), and also help enable the lower limb amputee provide a more cosmetically natural appearance under shoeless situations as well.

Thus in one embodiment, the invention may be a removable cosmetic skin covering device for a lower limb prosthetic, such as a prosthetic leg or foot (here the terms lower limb prosthetic and prosthetic foot will often be used interchangeably).

The device will enable the same prosthetic foot, without readjustment, to be used by an amputee with a prosthetic foot for either walking with shoes having shoe heels and other shoe elevation, or walking barefoot with minimal risk of knee hyperextension or prosthetic posterior lean.

The device will generally comprise a flesh colored skin (skin) covering configured to cover at least the foot portion of a prosthetic foot normally adjusted for walking with a shoe having at least a shoe heel with a shoe heel height of ⅜″ or more. This skin will have both a skin sole thickness in the foot sole region of said skin (often this will be relatively thin), as well as an internal heel or sole insert with an insert height, such that when the skin is applied to the prosthetic foot, the skin elevates at least the heel of the prosthetic foot to a sufficient height above the floor as to reduce the risk of knee hyperextension or prosthetic posterior lean when said amputee walks barefoot (i.e. without the shoe). The user will remove the covering when walking with shoes, but then rapidly apply the prosthetic covering when walking without shoes.

### BRIEF DESCRIPTION OF THE DRAWINGS

FIG. 1 shows the positions of the major parts of the legs during the stance and swing phases of the normal gait cycle. Note that even during the stance phase, the angle of the knee is normally less than 180°, thus allowing the knee of the stance (weight bearing) leg to bend inward slightly and absorb some of the shock of supporting weight.

FIG. 2 shows a common gait abnormality often caused by a misadjusted prosthetic leg, such as can happen when an amputee using a prosthetic leg adjusted for walking with shoes attempts to walk without the shoes. Here during stance phase, the angle of the knee becomes hyperextended beyond 180°. This can result in prosthetic posterior lean, and additionally (particularly if the walker retains a natural knee joint) puts unwanted stress on the amputee's already overstressed natural knee tissues.

FIG. 3 shows a detail of a prosthetic foot, prosthetic foot shell, and the invention's improved prosthetic foot covering. The invention's covering adjusts the elevation of the prosthetic foot to compensate for the missing shoe, while also providing a cosmetic cover that can be made to resemble the appearance of natural foot skin.

FIG. 4 shows a profile of an artificial foot with a foot shell, a profile of the same artificial foot now wearing the invention's improved prosthetic foot covering, and the differences between the two. This helps show, for example, the heel height adjustments produced by the invention's improved prosthetic foot covering.

FIG. 5 shows two alternate embodiments of the invention. In one embodiment, the covering extends only over the prosthetic foot shell, while in the other embodiment, the covering covers both the prosthetic foot shell and other elements of the prosthetic foot, thus producing a more natural “skin like” appearance overall.

FIG. 6 shows one mechanism by which the invention may act to reduce the problem of knee hyperextension.

FIG. 7 shows a low-cost method of producing the invention.

### DETAILED DESCRIPTION OF THE INVENTION

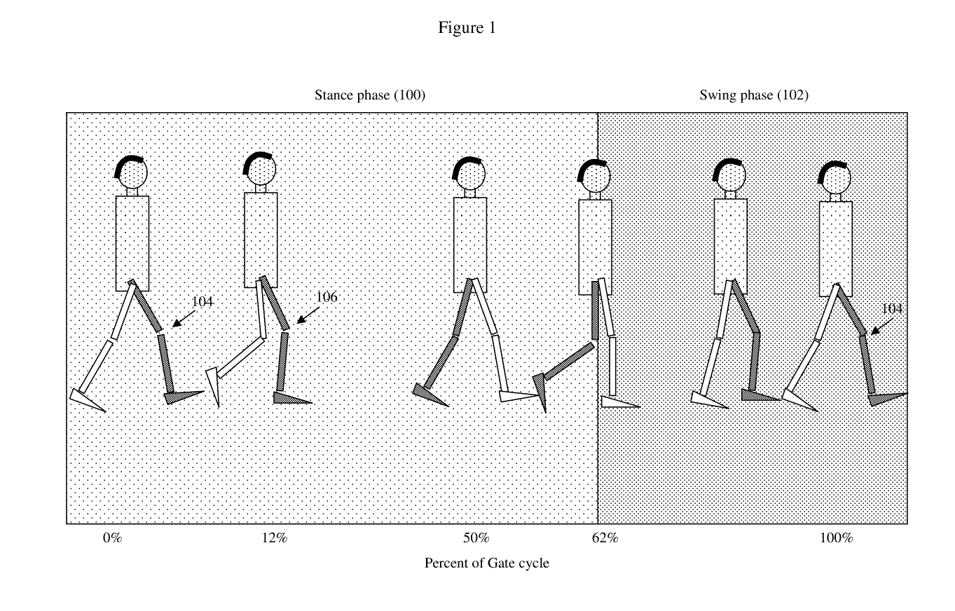

In order to better show the purpose and mode of operation of the invention, a simplified diagram of an ideal human gait cycle is shown in FIG. 1, which shows the positions of the major parts of the legs (e.g. the femur, the tibia, the knee, ankle and foot) during the stance (100) and swing (102) phases of the normal gait cycle. Note that in a proper or normal stance phase, the angle of the knee (104), (106) will be somewhat less than 180°, thus allowing the knee of the stance (weight bearing) leg to bend inward slightly, and absorb some of the shock of supporting the weight of the walkers body.

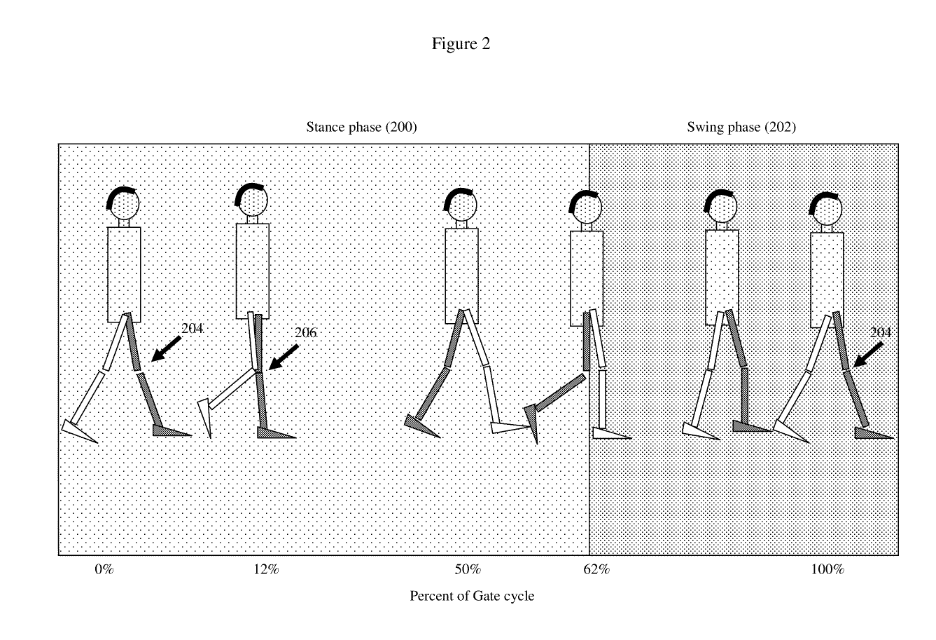

By contrast, FIG. 2 shows a common gait abnormality that is often caused by a misadjusted prosthetic leg. Here again, both stance phase (200) and swing phase (202) are shown. This problem frequently happens when an amputee using a prosthetic leg that is otherwise properly adjusted for walking with shoes attempts to walk without the shoes. Here during the stance phase (200), the angle of the knee becomes hyperextended beyond 180° (204), (206). This results in several problems, including prosthetic posterior lean. Additionally, if the amputee is a transtibial amputee with a functional natural knee joint, this knee hyperextension can put unnatural pressure on the amputee's natural knee tissue, resulting in higher risk for subsequent knee joint damage.

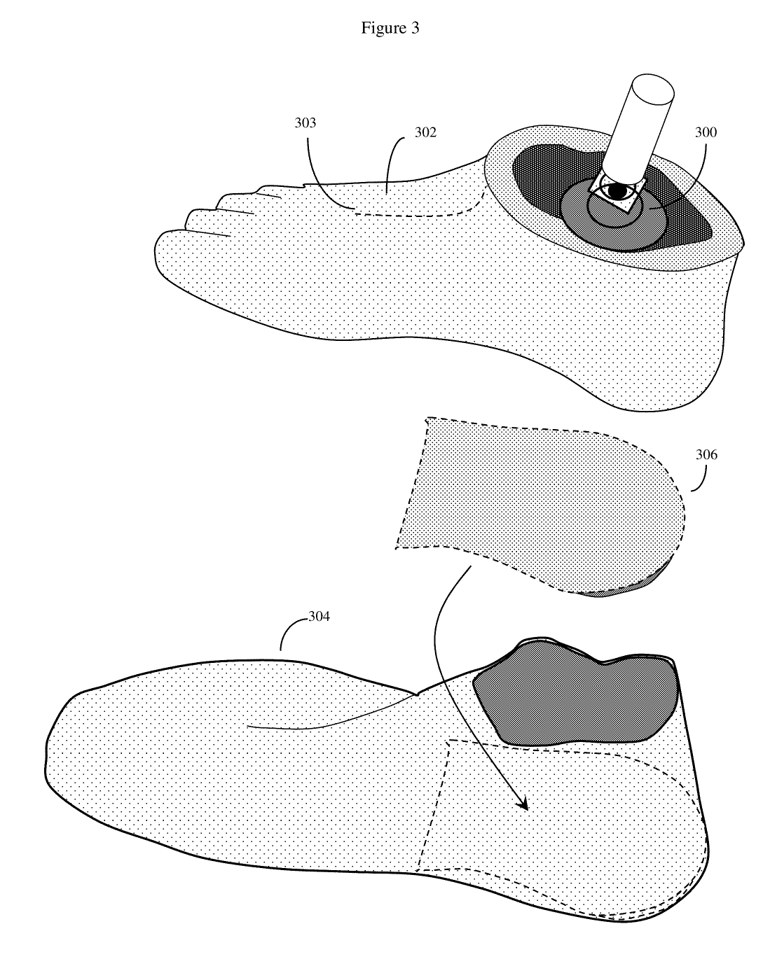

FIG. 3 shows a detail of a prosthetic foot (300), prosthetic foot shell (302), and the invention's improved prosthetic foot covering (304) which has at least an internal heel height insert (306) which often may extend to adjusting for the overall shoe height as well. In some embodiments, this may be an integral part of the foot covering (304). In other embodiments, this insert may be detachable. The invention adjusts the elevation of the prosthetic foot to compensate for the height of the missing shoe, and also provides a natural covering that can be made to resemble the appearance of natural foot skin.

As previously discussed, in a preferred embodiment, the invention's improved prosthetic foot covering is a removable flesh colored prosthetic covering that enables the same prosthetic foot, without readjustment, to be used by an amputee with either one or two prosthetic feet for (when the covering is off) walking with shoes, or walking without shoes (when the covering is on). Assuming that the prosthetic foot or feet are properly adjusted for walking with shoes, then the invention's covering automatically provides the proper height adjustment to the soles of the prosthetic foot or feet to enable the amputee to walk “barefoot” without encountering a higher risk for prosthetic posterior lean or knee hyperextension.

As previously discussed, that although heel height adjustments will primarily be used in this disclosure as an example of this type of shoe height adjustment, other types of shoe height adjustment, such as overall shoe sole height, may also be compensated for by embedding the proper type of insert into the prosthetic covering. Thus in this discussion, all shoes are presumed to have at least shoe heels with a shoe heel height of at least ⅜″, but these shoes may also have an overall sole height as well throughout, and the insert may include this overall sole height adjustment as well.

Also as previously discussed, in some embodiments, the internal heel and optional sole insert may be an integral part of the covering, in that it is inseparable from the covering. In other embodiments, the internal heel and optional sole insert may be attached to the covering after the covering is manufactured, and may even be user replaceable or adjustable as desired.

When the invention's skin or covering is applied to a prosthetic foot, the skin or covering, aided by the internal insert, thus elevates at least the heel of the prosthetic foot to a sufficient height above the floor as to reduce the risk of knee hyper extension or prosthetic posterior lean.

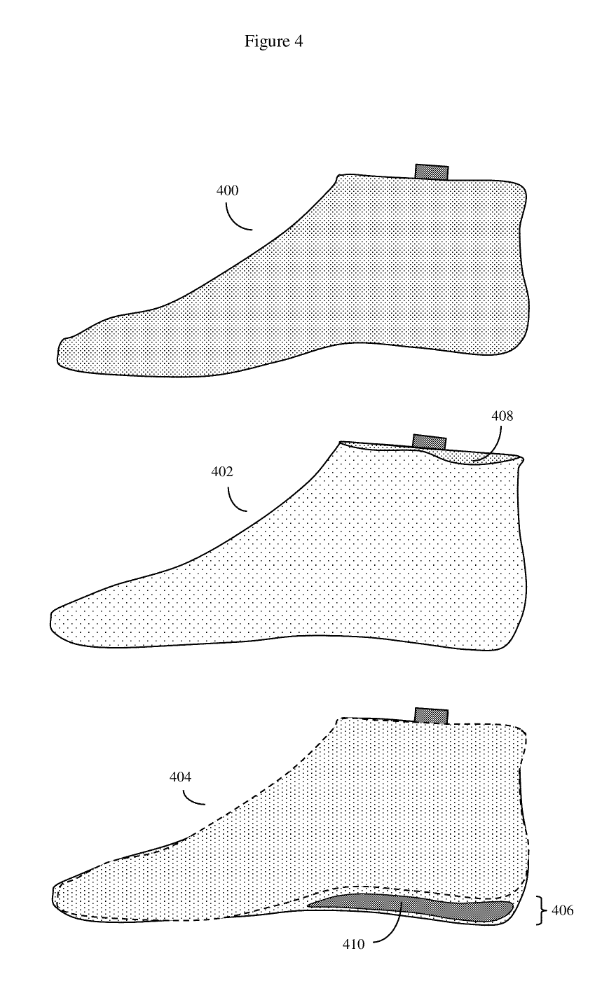

This effect is shown in FIG. 4. FIG. 4 shows a profile of an artificial foot shell (400), an artificial foot shell now wearing the invention's improved prosthetic foot covering (402), and the height differences between the two (404), thus illustrating at least the heel height adjustments produced by the invention's improved prosthetic foot covering (406). Here the prosthetic foot covering (402) is shown as not quite fully covering the top of the prosthetic foot shell, thus creating a small gap (408) exposing the prosthetic foot shell underneath the covering. The insert is also shown in cross section (410).

Note that from the outside, all an observer will see is an apparently natural looking foot (402). That is to improve the cosmetic appearance; the covering will be designed to minimize the visual impact of the internal insert and the sole of the covering from outside observers. Of course since there are natural color variations between the sole and heel of a natural human foot and the surrounding skin, the color of the covering or skin need not be totally uniform, but the general goal is, within the available cost budget, to strive to create a natural looking color pattern rather than, for example, making the sole of the covering a completely non-natural color.

The insert will often be made a semi-rigid but partially compressible material that returns to its natural shape and configuration during the swing phase of the amputee's gait. At a minimum, the insert will be a heel insert with a heel height of ⅜″ or higher, and at a maximum, the insert may additionally have an overall thickness throughout the sole region of the foot, and usually also have an additional heel height of ⅜″ or higher above this basic sole region as well. In this manner the insert can compensate for differences in overall shoe height as well as in shoe heel elevation.

Although, as will be discussed, in many embodiments the insert will be formed along with the rest of the skin or covering in the same molding process, and thus may be an integral part of the skin or covering (e.g. non-removable), in other embodiments, the insert may be removable, and indeed a plurality of inserts of different heights may be placed in said skin or covering.

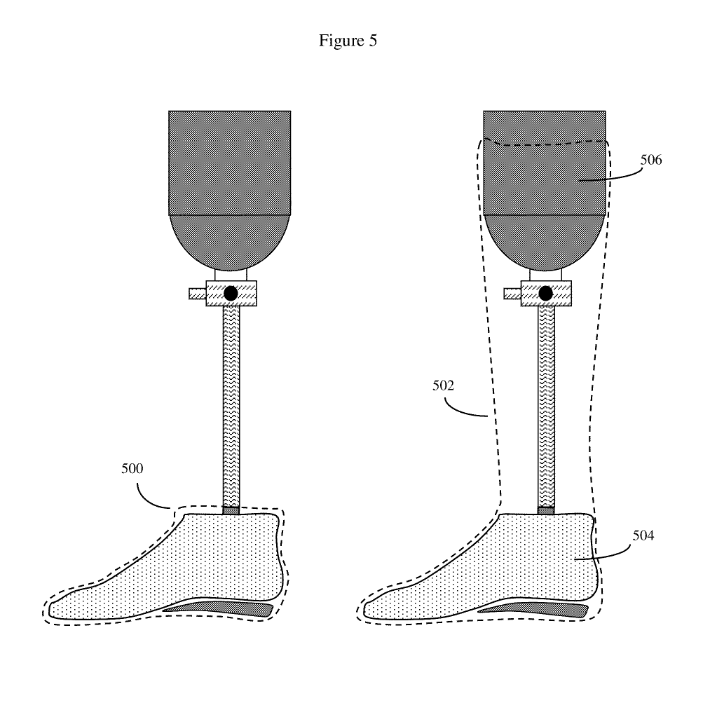

The invention's skin or covering may cover as little as the prosthetic foot shell, or as much as the entire prosthetic foot, or anywhere inbetween. For example, FIG. 5 shows two alternate embodiments of the invention. In one embodiment, the covering (500) extends only over the prosthetic foot shell, while in the other embodiment (502), the covering covers both the prosthetic foot shell, the shaft, the socket and other elements of the prosthetic foot, thus producing a more natural “skin like” appearance that covers nearly all of the prosthetic foot, from the prosthetic foot shell (504) to the prosthetic foot socket (506).

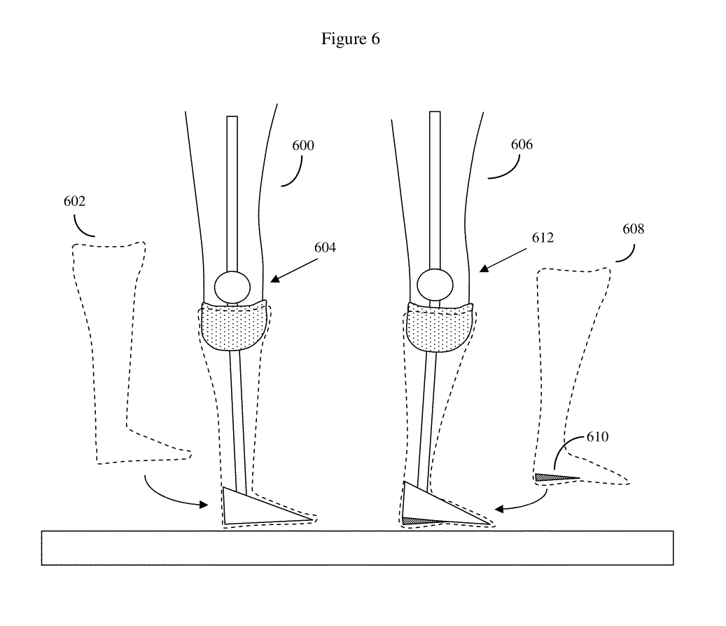

FIG. 6 shows one mechanism by which the invention may act to reduce the problem of knee hyperextension. In FIG. 6 (600), an transtibial amputee with a functional natural knee joint is attempting to walk barefoot using a prosthetic leg adjusted for walking with shoes, using only a prior art skin or covering without any height adjustment insert (602) As a result, some knee hyperextension (604) occurs during stance phase.

By contrast, in (606), the same amputee is walking with same prosthetic foot covered with the invention's improved covering (608) that now has a at least a heel height adjustment insert (610). Due to the heel height adjustment, during stance phase, the added heel height (and other heel height) helps position the amputee's foot at a more favorable angle, thus reducing the problem of knee hyperextension (612).

### Methods of Producing the Invention:

The invention may often be conveniently produced from a moldable synthetic polymer, such as silicone, polyurethane, synthetic rubber, or other natural polymer, synthetic polymer, or synthetic organic polymer. The covering may either be composed entirely of the moldable synthetic material, or often may comprise an embedded woven or nonwoven fabric coated or embedded in the moldable synthetic polymer to give the skin or covering extra tear resistance and elasticity.

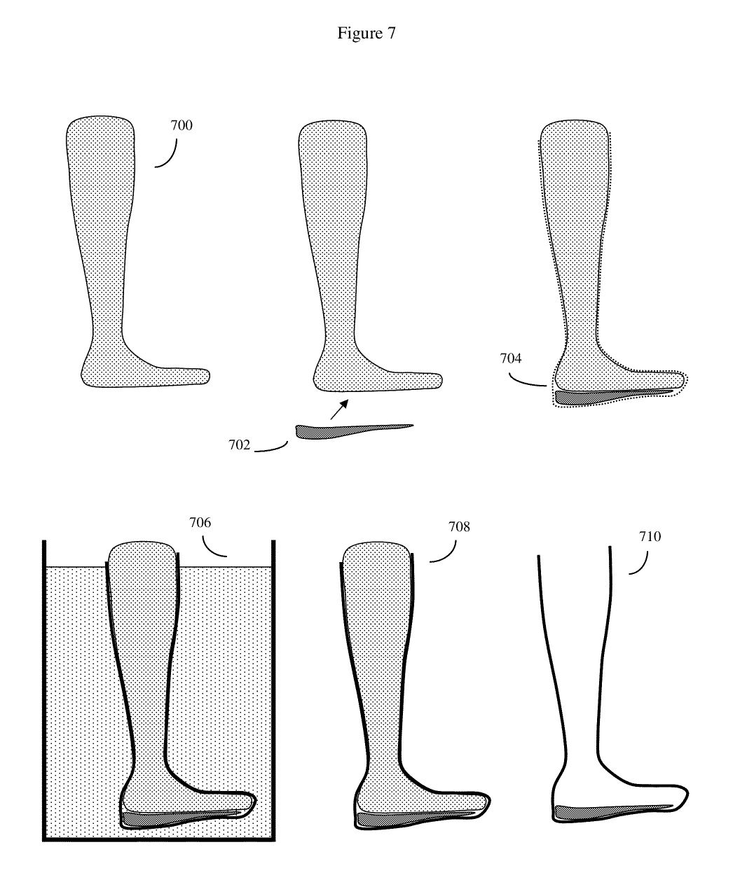

FIG. 7 shows a low-cost method of producing the invention. This method uses a single mold (700) or “last” configured in the shape of a typical prosthetic foot shell and lower foot region. An insert (702) may be placed on the sole of this mold or “last”, and as needed held in place by small snaps or other detents (not shown). In some embodiments, to provide a woven or nonwoven fabric inner core, the mold and detent may then be covered by smooth stocking-like woven or nonwoven fabric (704).

The mold or “last” may then be dipped into a liquid form of the moldable synthetic polymer (706), and then removed from the dip (708). The moldable synthetic polymer will harden, forming the skin or covering, with the insert held firmly inside the outer layer of the synthetic polymer. Once hardened, the skin or covering can be stripped from the outer surface of the mold (710). This skin or covering can then be further processed, for example by cutting appropriate slits and adding suitable fasteners (e.g. zippers, hook and clasp fasteners, and the like, preferably durable, inconspicuous, and flesh colored) as desired to allow the user to rapidly put on and take off the covering in use.

Many other types of molding process, as well as more complex molds (e.g. injection molding between both an inner and outer mold, etc.) may also be used. The net result will be to produce a covering or skin where the internal heel or sole insert is positioned inside the outer surface of the moldable synthetic polymer so that the sole of said skin has a natural skin color that is substantially similar to other portions of said skin.

In some embodiments, either as part of the molding process, or as a secondary operation afterward, the outside of the skin or covering can be further differentially colored or textured so as to increase the similarity of appearance between the outside of the skin and either the skin of the amputee's natural foot (if the amputee has a natural foot), or the appearance of a typical natural foot if the amputee does not have a natural foot.

### Using the Device

When the user is walking with shoes and one or more prosthetic legs configured for shoes, the invention's covering will usually not be worn, but rather may be inconspicuously kept by the user (e.g. in the user's pockets, purse, suitcase, etc.). When the user encounters a situation where socially, the appearance of waking barefoot is desirable (for example when entering a home where shoes are not worn), the user will then apply the skin or covering to the prosthetic foot or feet, and then will be able to walk “barefoot” with minimal risk of knee hyperextension or prosthetic posterior lean, and with minimal social embarrassment.

The device may be made commercially in a range of different sizes and insert types. In some situations, in order to insure the best quality fit, it may be useful to first analyze the user's walking gate patterns under various conditions, and use these walking gate patterns to select various parameters such as the height or shape of the insert and/or the compressibility or other characteristics of the insert material.

### Other Embodiments

As previously discussed, the mechanical portions of the artificial foot are usually covered by a foot shell, which is a relatively thick, skin-colored plastic covering, molded in the general shape of a natural foot (i.e. often with toes) designed to allow the artificial foot to fit into the shoe that the lower limb amputee intends to wear. At present, foot shells are generally designed for very secure attachment to the mechanical portions of the artificial foot, and it takes a considerable amount of effort to attach or remove a foot shell from the mechanical portions of the artificial foot.

In principle, however, the foot shell could be redesigned for easier attachment and detachment from the underlying mechanisms of the artificial foot, for example by cutting a slit (FIG. 3 (303) in the foot shell to make it easier to deform while taking on and off.

In an alternative embodiment of the invention, the removable cosmetic skin covering device for a prosthetic foot, enabling the same prosthetic foot, without readjustment to be used by an amputee with a prosthetic foot for either walking with shoes having shoe heels or walking barefoot with minimal risk of knee hypextension or prosthetic posterior lean may instead comprise a flesh covered foot shell that is configured to cover the mechanical portions of at least the prosthetic foot. In this case, one version of the foot shell would be configured for walking with shoes, while an alternate version of the foot shell would have an internal heel or sole insert with an insert height, such that when the foot shell is applied to the mechanical portions of the prosthetic foot, the foot shell elevates at least the heel of the prosthetic foot to a sufficient height above the floor as to reduce the risk of knee hyperextension or prosthetic posterior lean when the amputee walks barefoot.

Alternatively, the alternate version of the foot shell configured for walking barefoot could have a very thick heel and possibly thick sole as well, so as to elevate at least the heel of the prosthetic foot to a sufficient height above the floor as to reduce the risk of knee hyperextension or prosthetic posterior lean when the amputee walks barefoot.

1. A removable cosmetic skin covering device for a prosthetic foot, said device enabling the same prosthetic foot, without readjustment, to be used by an amputee with a prosthetic foot for either walking with shoes having shoe heels or walking barefoot with minimal risk of knee hyperextension or prosthetic posterior lean, said device comprising:
a flesh colored skin (skin) configured to cover at least said prosthetic foot;
said prosthetic foot adjusted for walking with a shoe having a shoe heel with a shoe heel height of ⅜″ or more;
said skin having a skin sole thickness in the foot sole region of said skin;
said skin having an internal heel or sole insert with an insert height;
wherein when said skin is applied to said prosthetic foot, said skin elevates at least the heel of said prosthetic foot to a sufficient height above the floor as to reduce the risk of knee hyperextension or prosthetic posterior lean when said amputee walks barefoot.

2. The device of claim 1, wherein said amputee is a transtibial amputee with a functional natural knee joint, and said device prevents hyperextension of said functional natural knee joint.

3. The device of claim 1, wherein said skin comprises a moldable synthetic polymer, and wherein said internal heel or sole insert is positioned inside the outer surface of said moldable synthetic polymer so that the sole of said skin has a natural skin color that is substantially similar to other portions of said skin.

4. The device of claim 1, wherein said moldable synthetic polymer is selected from a group consisting of silicon, polyurethane, synthetic rubber, other natural polymer, synthetic polymer, or synthetic inorganic polymer.

5. The device of claim 1, wherein said skin comprises a stretchable synthetic woven or nonwoven fabric.

6. The device of claim 1, wherein the outside of said skin is further differentially colored or textured so as to increase the similarity of appearance between the outside of said skin and either the skin of said amputee's natural foot if said amputee has a natural foot, or the appearance of a natural foot if said amputee does not have a natural foot.

7. The device of claim 1, wherein said skin further comprises at least one flesh colored fastener configured to enable said skin to be rapidly mounted and removed from said prosthetic foot.

8. The device of claim 1, wherein said insert is formed from a semi-rigid but partially compressible material that returns to its natural shape and configuration during the swing phase of the amputee's gait.

9. The device of claim 1, wherein said insert is non-removable.

10. The device of claim 1, wherein said insert is removable, and wherein a plurality of inserts of different heights may be placed in said skin.

11. The device of claim 1, wherein said insert has a heel height of ⅜″ or higher.

12. The device of claim 1, wherein said prosthetic foot further comprises a prosthetic shaft extending above said prosthetic foot, and a socket, and wherein said skin is configured to also cover said prosthetic shaft and at least portions of said socket.

13. A method of enabling an amputee with a prosthetic foot to minimize the risk of knee hyperextension or prosthetic posterior lean while using said prosthetic foot, without readjustment, to walk with either shoes having shoe heels or barefoot, said method comprising:
attaching a prosthetic foot adjusted for walking with shoes having shoe heels with a shoe heel height of ⅜″ or more to the amputated leg of said amputee;
providing a removable cosmetic skin covering device for said prosthetic foot, said device comprising:
a flesh colored skin (skin) configured to cover at least said prosthetic foot;
said skin having a skin sole thickness in the foot sole region of said skin;
said skin having an internal heel or sole insert with an insert height;
wherein when said skin is applied to said prosthetic foot, said skin elevates at least the heel of said prosthetic foot to a sufficient height above the floor as to reduce the risk of knee hyperextension or prosthetic posterior lean when said amputee walks barefoot;
removing said skin from said prosthetic foot when said amputee desires to walk with shoes;
and applying said skin to said prosthetic foot when said amputee wishes to walk barefoot.

14. The method of claim 13, wherein said amputee is a transtibial amputee with a functional natural knee joint, and said device prevents hyperextension of said functional natural knee joint.

15. The method of claim 13, wherein said skin comprises a moldable synthetic polymer, and wherein said internal heel or sole insert is positioned inside the outer surface of said moldable synthetic polymer so that the sole of said skin has a natural skin color that is substantially similar to other potions of said skin.

16. The method of claim 13, wherein said moldable synthetic polymer is selected from a group consisting of silicone, polyurethane, synthetic rubber, other natural polymer, synthetic polymer, or synthetic inorganic polymer.

17. The method of claim 13, wherein said skin comprises a stretchable synthetic woven or nonwoven fabric.

18. The method of claim 13, further differentially coloring or texturing the outside of said skin so as to increase the similarity of appearance between the outside of said skin and either the skin of said amputee's natural foot if said amputee has a natural foot, or the appearance of a natural foot if said amputee does not have a natural foot.

19. The method of claim 13, further configuring said skin with at least one flesh colored fastener configured to enable said skin to be rapidly mounted and removed from said prosthetic foot;
opening said fastener in order to facilitate removal of said skin from said prosthetic foot, and
closing said fastener in order to facilitate application of said skin to said prosthetic foot.

20. The method of claim 13, wherein said insert is formed from a semi-rigid but partially compressible material that returns to its natural shape and configuration during the swing phase of the amputee's gait.

21. The method of claim 13, wherein said prosthetic foot further comprises a prosthetic shaft extending above said prosthetic foot, and a socket, and wherein said skin is configured to also cover said prosthetic shaft and at least portions of said socket.

22. The method of claim 13, further analyzing the walking gait patterns of said amputee, and using said walking gate patterns to select the height of said insert and/or the compressibility of said insert material.
